(19) United States
(12) Patent Application Publication
(10) Pub. No.: US 2010/0158857 A1
(43) Pub. Date: Jun. 24, 2010

(54) Compositions and methods for the inhibition of endothelial nitric oxide synthase activity

(75) Inventors: Wolfram Samlowski, Salt Lake City, UT (US); Muralidhar Kondapaneni, Salt Lake City, UT (US); John McGregor, Salt Lake City, UT (US)

Correspondence Address: THE MCCALLUM LAW FIRM, P. C., 685 BRIGGS STREET, PO BOX 929, ERIE, CO, 80516, US

(73) Assignee: University Of utah Research Foundation

(21) Appl. No.: 12/227,543

(22) PCT Filed: May 23, 2007
(86) PCT No.: PCT/US07/12410

§ 371 Date: Dec. 15, 2009

Related U.S. Application Data
(60) Provisional application No. 60/802,916, filed on May 23, 2006.

(51) Int. Cl. A61K 38/20; A61K 31/198; A61K 31/221; A61K 31/155; A61K 31/416; A61K 31/17; A61K 31/195; A61P 35/00

(52) U.S. Cl. 424/85.2; 514/565; 514/551; 514/634; 514/406; 514/580; 514/562; 514/561

(57) ABSTRACT

The present invention relates to the treatment and prevention of the toxic effects associated with increased nitric oxide synthase activity in endothelial cells. In particular, the present invention relates to compounds and methods of treatment that inhibit the nitric oxide synthases present in endothelial cells and methods for treating diseases using such compounds and methods.

### CROSS REFERENCE TO RELATED APPLICATION

This Application claims priority from U.S. Provisional Patent Application No. 60/802,916 filed May 23, 2006 entitled, “Methods and Compounds for the Inhibition of eNOS Activity,” which application is hereby incorporated by reference in this application in its entirety.

### FIELD OF THE INVENTION

The present invention relates to the field of treatment and prevention of the toxic effects associated with increased nitric oxide synthase activity in endothelial cells. In particular, the present invention relates to compounds and methods of treatment that inhibit the nitric oxide synthases present in endothelial cells and methods for treating diseases using such compounds and methods.

### BACKGROUND OF THE INVENTION

Interleukin-2 (IL-2) is useful for the treatment of cancer. Presently, it is approved for the treatment of both metastatatic renal cell carcinomas and metastatic melanomas. Unfortunately, its use is restricted due to severe side effects that include fever, nausea, hypotension and vascular leak syndrome (VLS).

Although it has been established that nitric oxide synthesis is strongly induced by IL-2 treatment in humans and mice, the mechanism of IL-2 induced cardiovascular toxicity is not completely understood. Unfortunately, although general inhibitors of nitric oxide synthase (NOS) such as N^{G}-monomethyl-L-arginine (MLA), are able to inhibit IL-2 induced VLS, treatment with such agents has proven unsuitable due to excessive toxicity to the patient. (Samlowski et al 1995, J Immunother 18:166) Accordingly, compounds and methods which allow for administration of high doses of IL-2 without creating the negative side effects, such as hypotension and VLS, are greatly needed.

### SUMMARY OF THE INVENTION

The present invention is based on the observation that there appear to be negative effects associated with increased nitric oxide synthase activity in endothelial cells. As such, targeted delivery of general nitric oxide synthase inhibitors and/or endothelial nitric oxide synthase (eNOS) inhibitors directly to endothelial cells is a novel way of decreasing such toxic effects, such as hypotension and vascular leak syndrome (VLS). This was a surprising result as general nitric oxide synthase inhibitors had previously been found to be toxic when administered to patients. Herein, the present invention provides for modifications to general nitric oxide synthase inhibitors in order to provide for targeting to the endothelial cells of a mammal. The present invention also provides for eNOS inhibitors that are also capable of inhibiting nitric oxide synthase activity in the endothelial cells of a mammal.

In accordance with the purpose(s) of this invention, as embodied and broadly described herein, this invention, in one aspect, relates to compounds and methods for inhibiting nitric oxide synthase in endothelial cells.

In one embodiment the present invention relates to compositions and methods for treating disease or conditions related to increased activity of nitric oxide synthase activity in endothelial cells.

In another embodiment, the nitric oxide synthase inhibitors of the present invention are modified general nitric oxide synthase inhibitors or eNOS inhibitors.

In a particular embodiment, the modified general nitric oxide synthase inhibitors are based on a molecule selected from arginine-based analogues, methylated arginines, substituted L-arginine, nitro-arginine, L-N^{G}-nitroarginine, N^{G}-mono-methyl-L-arginine (L-NMMA), N-nitro-L-arginine methyl ester (L-NAME), N-amino-L-arginine, N-methyl-L-arginine, N^{G}-monomethyl-L-arginine (L-NMA), N^{G}-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, or mixtures thereof.

In another particular embodiment, such modified general nitric oxide synthase inhibitor is converted into a polymeric form or conjugated to one or more polyethylene glycol molecules. One such example of a polymeric form is (N^{G}-mono-methyl-L-arginine)⁷. One such example of a general nitric oxide inhibitor conjugated to polyethylene glycol is N^{G}-mono-methyl-L-arginine.

In a particular embodiment, such eNOS inhibitor is selected from asymmetric dimethylarginine or N(5)-(1-iminoethyl)-L-ornithine (L-NIO).

The compositions and methods of the present invention may further provide for the inclusion or co-administration of a pharmaceutically effective amount of a nitric oxide scavenger or an interleukin. In a particular embodiment, such interleukin is interleukin-2.

In a particular embodiment, the pharmaceutically effective dose of interleukin is increased due to the inhibition of nitric oxide synthase activity in said endothelial cells.

In another particular embodiment, the toxic effects of interleukin therapy are lessened due to the inhibition of nitric oxide synthase activity in said endothelial cells.

The diseases or conditions capable of being treated with the compositions or methods of the present invention may be selected from hypotension, septic shock, vascular leak syndrome, cancer, sepsis, acidosis, spinal anesthesia, administration of inhalation agents, administration of nitrate preparations, administration of calcium channel blockers or administration of ACE inhibitors.

### DETAILED DESCRIPTION OF THE INVENTION

The present invention may be understood more readily by reference to the following detailed description of particular embodiments of the invention.

Particular advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

Before the present compositions and/or methods are disclosed and described, it is to be understood that this invention is not limited to specific compositions or methods, as such may, of course, vary, unless it is otherwise indicated. It is also to be understood that the terminology used herein is for the purpose of describing particular embodiments only and is not intended to be limiting.

### Definitions:

For the purposes of the present invention, the following terms shall have the following meanings:

For the purposes of the present invention, the term “a” or “an” entity refers to one or more of that entity; for example, “a compound” or “a molecule” refers to one or more of those elements or at least one element. As such, the terms “a” or “an”, “one or more” and “at least one” can be used interchangeably herein. It is also to be noted that the terms “comprising,” “including,” and “having” can be used interchangeably. Furthermore, an element or means “selected from the group consisting of” refers to one or more of the elements in the list that follows, including mixtures (i.e. combinations) of two or more of the elements.

For the purposes of the present invention, the term, “nitric oxide scavenger” refers to any molecule capable of inactivating one or more nitric oxide molecules.

For the purposes of the present invention, the term, “treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

For the purposes of the present invention, the term, “therapeutically effective amount” refers to an amount effective, at dosages and for periods of time necessary, to achieve the desired therapeutic result. A therapeutically effective amount of a modified general nitric oxide synthase inhibitor, for instance, may vary according to factors such as the disease state, age, sex, and weight of the individual, and the ability of the antibody to elicit a desired response in the individual. A therapeutically effective amount is also one in which any toxic or detrimental effects of the compound are outweighed by the therapeutically beneficial effects.

For the purposes of the present invention, the term, “mammal” refers to any animal classified as a mammal, including humans, domestic and farm animals, and zoo, sports, or pet animals, such as dogs, cats, cows, horses, sheep, pigs, etc. In a particular embodiment, such mammal is human.

For the purposes of the present invention, the term administration “in combination with” or “co-administration with” one or more further therapeutic agents includes simultaneous (concurrent) and consecutive administration in any order.

Pathologic conditions and disorders “associated with” increased nitric oxide synthase activity in endothelial cells or “characterized by” increased nitric oxide synthase activity in endothelial cells and/or wherein increased nitric oxide synthase activity in endothelial cells is an “important regulator” are those conditions and disorders where nitric oxide excess in the endothelial cells of a mammal is correlated with a disease or condition. Such disorders and conditions include, for example, hypotension, septic shock, vascular leak syndrome, cancer, sepsis, acidosis, spinal anesthesia, administration of inhalation agents, administration of nitrate preparations, administration of calcium channel blockers or administration of ACE inhibitors.

For the purposes of the present invention, ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

Reference will now be made in detail to particular embodiments of the invention.

### Inhibitors of Nitric Oxide Synthase

Nitric oxide is produced from the conversion of L-arginine to citrulline by nitric-oxide synthase of which there appear to be three isoforms. The three isoforms include endothelial nitric oxide synthase (eNOS), neuronal nitric oxide synthase (nNOS) and inducible nitric oxide synthase (iNOS). The present invention is directed to compositions and methods that cause the inhibition of nitric oxide synthases present in endothelial cells.

In one embodiment, modified general nitric oxide synthase inhibitors are provided for inhibition of nitric oxide synthase activity in the endothelial cells of a mammal. Such modified general nitric oxide synthase inhibitors are capable of inhibition of one or more isoforms of nitric oxide synthase in the endothelial cells of a mammal.

In another embodiment, eNOS inhibitors are provided for inhibition of nitric oxide synthase activity in the endothelial cells of a mammal. Such eNOS inhibitors are capable of inhibiting only the eNOS isoform of nitric oxide synthase.

The modified general nitric oxide inhibitors of the present invention include any compound capable of preventing the conversion of L-arginine to citrulline. In a particular embodiment, such modified general inhibitors are selected from arginine-based analogues, methylated arginines, substituted L-arginine, nitro-arginine, L-N^{G}-nitroarginine, N^{G}-mono-methyl-L-arginine (L-NMMA), N-nitro-L-arginine methyl ester (L-NAME), N-amino-L-arginine, N-methyl-L-arginine, N^{G}-monomethyl-L-arginine (L-NMA), N^{G}-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, or mixtures thereof.

The present invention provides for modified forms of general nitric oxide synthase inhibitors in order to provide for targeting of endothelial cells. Such modification may include any modification that increases the size or bulkiness of the inhibitor in order to provide for delivery to the endothelial cells of a mammal. In one embodiment, such modification includes the conversion of a general nitric oxide synthase inhibitor into a polymeric form or the attachment of polyethylene glycol to such inhibitor. In a particular embodiment, the conversion of a general nitric oxide synthase inhibitor into a polymeric form is (N^{G}-mono-methyl-L-arginine)⁷. In another particular embodiment, the conversion of a general nitric oxide synthase inhibitor into a conjugated form with polyethylene glycol is selected from N^{G}-mono-methyl-L-arginine or mono-methyl-L-arginine

The eNOS inhibitors of the present invention are any inhibitor capable of inhibiting only the eNOS isoform of nitric oxide synthase. In a particular embodiment, such specific inhibitor is selected from eNOS inhibitor is asymmetric dimethylarginine or N(5)-(1-iminoethyl)-L-ornithine(L-NIO).

### Nitric Oxide Scavengers

The present invention may further provide for the administration of a nitric oxide scavenger in combination with nitric oxide synthase inhibitors and/or interleukin. In a particular embodiment, the nitric oxide scavenger is selected from redox dyes; methylene blue; heme binders; superoxide dismutase; mimetics of superoxide dismutase, such as M40403; PTIO (2-phenyl-4,4,5,5-tetramethyl-imidazoline-1-oxyl3-oxide); Carboxy-PTIO (2-(4-Carboxyphenyl)-4,4,5,5-tetramethylimidazoline-1-oxyl-3-oxide); AMD6221 (ruthenium[hydrogen(diethylenetrinitrilo) pentaacetato] chloride); Dithiocarbamate; or Vitamin B12, including cobalamin or some of its derivatives such as cobinamide, hydroxocobalamin and the like.

### Interleukin

The present invention provides for co-administration of an interleukin in combination with nitric oxide synthase inhibitors. The interleukin of the present invention may be any interleukin with a therapeutic effect. In a particular embodiment, such interleukin is interleukin-2.

In one embodiment, the pharmaceutically effective dose of interleukin administered to a mammal is higher due to the administration of nitric oxide synthase inhibitors to endothelial cells.

In another embodiment, the toxic effects of interleukin therapy are lessened due to the administration of a modified general nitric oxide synthase inhibitor and/or eNOS inhibitor to endothelial cells.

### Treatment of Disease

The present invention provides for compositions and methods of treating a mammal with modified general nitric oxide synthase inhibitors or eNOS inhibitors. Such treatment may further be given in combination with interleukin. The methods and compositions for such treatment may further provide for co-administration with a nitric oxide scavenger.

The diseases capable of being treated by the compositions and methods of the present invention include hypotension, septic shock, vascular leak syndrome, cancer or any condition associated with the same. The diseases may also include any condition or disorder associated with or characterized by endothelial nitric oxide production and/or dysfunction.

The present invention further provides compositions and methods of treating or preventing a disease associated with excessive vasodilation. In a particular embodiment, such condition associated with excessive vasodilation is selected from sepsis, acidosis, spinal anesthesia, administration of inhalation agents, administration of nitrate preparations, administration of calcium channel blockers or administration of ACE inhibitors. Such condition or disorder may be associated with hypotension, septic shock and/or any other type of related condition.

The compositions of the present invention may be given prior to administering an agent known to cause hypotension, septic shock, vasodilation and the like in order to prevent such condition from occurring in a mammal.

The compositions and methods of the present invention may be used in a mammal suffering from cancer. In one embodiment, treatment of such mammal may be in combination with an interleukin. In a particular embodiment, such interleukin is interleukin-2. In another embodiment, treatment of such mammal may further include co-administered of a nitric oxide scavenger.

### Pharmaceutical Compositions and Routes of Administration

Aqueous compositions of the present invention include a therapeutically effective amount of modified general nitric oxide synthase inhibitors and/or eNOS inhibitors, and the like, dissolved or dispersed in a pharmaceutically acceptable carrier or aqueous medium. Aqueous compositions may further include interleukin or nitric oxide scavengers. Aqueous compositions of gene therapy vectors expressing any of the foregoing are also contemplated. The phrases “pharmaceutically or pharmacologically acceptable” refer to molecular entities and compositions that do not produce an adverse, allergic or other untoward reaction when administered to an animal, or a human, as appropriate.

Aqueous compositions of the present invention include a therapeutically effective amount of the compound, dissolved or dispersed in a pharmaceutically acceptable carrier or aqueous medium. Such compositions can also be referred to as inocula. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. For human administration, preparations should meet sterility, pyrogenicity, general safety and purity standards as required by FDA Office of Biologics standards.

The biological material should be extensively dialyzed to remove undesired small molecular weight molecules and/or lyophilized for more ready formulation into a desired vehicle, where appropriate. The active compounds will then generally be formulated for parenteral administration, e.g., formulated for injection via the intravenous, intramuscular, sub-cutaneous, intralesional, or even intraperitoneal routes. The preparation of an aqueous composition that contains an active component or ingredient will be known to those of skill in the art in light of the present disclosure. Typically, such compositions can be prepared as injectables, either as liquid solutions or suspensions; solid forms suitable for use in preparing solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A therapeutic agent can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The use of peptide therapeutics as active ingredients is well-known in the art.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above; as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly, concentrated solutions for direct injection is also contemplated, where the use of DMSO as solvent is envisioned to result in extremely rapid penetration, delivering high concentrations of the active agents to a small area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580).

The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the protection desired. The person responsible for administration will, in any event, determine the appropriate dose for the individual subject.

The active therapeutic agents may be formulated within a mixture to include about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g., tablets or other solids for oral administration; liposomal formulations; time release capsules; and any other form currently used.

One may also use nasal solutions or sprays, aerosols or inhalants in the present invention. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal preparations are known and include, for example, antibiotics and antihistamines and are used for asthma prophylaxis.

Additional formulations which are suitable for other modes of administration include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%.

Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tables, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such therapeutically useful compositions is such that a suitable dosage will be obtained.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

### EXAMPLES

It should be appreciated by those skilled in the art that the techniques disclosed in the examples which follow represent techniques discovered by the inventors to function well in the practice of the invention, and thus can be considered to constitute particular modes for its practice. However, those of skill in the art should appreciate, in light of the present disclosure, that many changes can be made in the specific embodiments disclosed herein which will still obtain a like or similar result without departing from the spirit and scope of the invention.

### Example 1

### Induction of Hypotension and VLS in Wild-type and iNOS Knockout Mice

Groups of wild type (WT) and iNOS^{-/-} C57BL/6 mice (four per group) were treated with 800,000 IU IL-2 (i.p, b.i.d.) for 5 days. Hypotension and VLS were evaluated at the end of IL-2 treatment as follows.

Blood Pressure Evaluation:

Systolic blood pressure was measured via tail cuff method (Stoelting, Wood Dell, Ill.) using PowerLab digital signal transducer (AD instruments, Mountain View, Calif.). Systolic blood pressure was measured 2-4 hours after the last dose of IL-2. The lower limit of blood pressure detectable using this instrumentation is about 40 mm of Hg. This value of blood pressure was assigned whenever no detectable value could be obtained.

VLS Evaluation

Approximately 100,000 cpm of [¹²⁵l]-labeled albumin (specific activity 1-5 μCi/μg, dissolved in 0.1 ml 0.9N sterile saline) was injected intravenously into tail veins of mice, 2 hours after the last dose of IL-2. Mice were sacrificed at 6 hours after this dose, as preliminary studies indicated that this time interval resulted in maximal VLS. Peritoneal fluid was collected using blotting paper. Liver, spleen, intestines, kidneys, lungs and heart were also excised and analyzed for [¹²⁵l]-albumin accumulation. Data was expressed as percentage of radiolabeled albumin recovered from each tissue with the total amount recovered from each animal. This form of analysis minimizes differences induced by variability in injection technique.

Both the iNOS^{-/-} mice and the WT controls demonstrated a marked drop in systolic blood pressure. Analysis of radiolabeled albumin accumulation showed that WT and iNOS^{-/-} mice also developed VLS following IL-2 treatment. [¹²⁵l] labeled albumin retention in peritoneal fluid increased from 0.4±0.1% at baseline to 1.8±0.8% in iNOS WT mice and from 0.3±0.06% at baseline to 2.9±1.3% in iNOS^{-/-} mice following IL-2 treatment course (p<0.01). These results indicate iNOS is not required for the development of IL-2 induced hypotension or VLS.

### Example 2

### Effect of M40403 on IL-2 Induced Hypotension and VLS in iNOS^{-/-} Mice

IL-2 induced hypotension has been shown to be a consequence of superoxide and/or peroxynitrite induced oxidation of catecholamines. Therefore the SOD mimetic, M40403, was tested to see if it could reverse hypotension in iNOS^{-/-} mice following IL-2 treatment. Groups of mice (four per group) were treated with either IL-2 or IL-2 plus M40403 for five days. Systolic blood pressures dropped from a baseline 90±10 mm of Hg to 40±3 mm of Hg in IL-2 treated iNOS^{-/-} mice. Knock out mice treated with both IL-2 and M40403 had nearly complete reversal of hypotension, with restoration of systolic BP to 85±5 mm of Hg. This experiment verified that superoxide mediated mechanisms were responsible for the IL-2 induced hypotension even in iNOS^{-/-} mice. M40403 did not reverse IL-2 induced radio-albumin accumulation in peritoneal fluid in iNOS WT or iNOS^{-/-} mice. Thus superoxide does not appear to play a significant role in the pathogenesis of IL-2 induced VLS.

### Example 3

### Effect of 1400 W on IL-2 Induced Hypotension and Vascular Leak

The effects of the iNOS specific inhibitor 1400 W in IL-2 treated mice were tested. 1400 W was dissolved at a concentration of 1M in sterile PBS and administered via continuous subcutaneous infusion by Alzet mini osmotic pumps (Model 2001, Alzet Corporation, Cupertino, Calif.). Each pump contained 225 μL of solution that released a constant volume of 28 μL/day, resulting in a calculated delivery of 7 mg 1400 W/mouse/day. The pumps were implanted subcutaneously on the backs of mice using sterile surgical technique one day prior to the start of IL-2 treatment, under methoxyflurane (inhalant) anesthesia.

1400 W did not appear to reverse either hypotension or vascular leak in IL-2 treated mice. Systolic blood pressure in mice treated with IL-2 plus 1400 W remain depressed, similar to levels seen in mice treated with IL-2 alone (45±7 mm of Hg and 40 mm of Hg respectively). 1400 W also did not inhibit VLS. These results further verified that a functional iNOS enzyme does not appear to be required for IL-2 induced hypotension and vascular leak.

### Example 4

### Effects of MLA on IL-2 Induced Hypotension and VLS

To establish whether NO synthesis from other NOS isoforms (e.g. endothelial and neuronal NOS) contributed to IL-2 induced side effects, iNOS WT and iNOS^{-/-} C57BL/6 mice (four per group) were treated with either IL-2 alone or IL-2 with continuous subcutaneous administration of the general NOS inhibitor, N^{G}-monomethly-L-arginine (MLA) using Alzet mini pumps. MLA was dissolved in sterile ultra-filtered water at a concentration of 3.38M and loaded into pre-equilibrated osmotic pumps containing 225 μL of solution that released a constant volume of 28 μL/day, resulting in a calculated delivery of 18.5 mg MLA/mouse/day.

Following IL-2 administration, the iNOS^{-/-} mice were found to be severely hypotensive (40±5 mm of Hg) whereas iNOS^{-/-} mice that received MLA along with IL-2 had a restoration of systolic BP to a near baseline level of 80±10 mm of Hg.

MLA administration also inhibited IL-2 induced [¹²⁵l] albumin accumulation in peritoneal fluid in both iNOS WT and iNOS^{-/-} mice. Following IL-2 treatment, the amount of [¹²⁵l] labeled albumin accumulation in peritoneal fluid was 1.80±0.79% and 2.99±1.33% in WT mice and knock out mice, respectively. Retention of albumin was reduced to near baseline levels (0.562±0.24% and 1.66±0.06% respectively in iNOS WT and iNOS^{-/-} mice) following MLA administration during IL-2 treatment. These experimental results strongly support a significant role of eNOS and/or nNOS in the development of VLS following IL-2 administration.

### Example 5

### Design of Modified General Nitric Oxide Synthase Inhibitors

Several molecules were produced including a 7-mer polymer of NG-mono-methyl-L-arginine (i.e. [NG-mono-methyl-L-arginine]⁷), a PEG-conjugated NG-mono-methyl-L-arginine, and a PEG-conjugated mono-methyl-L-arginine conjugated. All were retained in the vasculature due to their size as evidenced by (what?)

### Example 6

### Polymethylarginine Inhibition of NO Synthesis

N^{G}-monomethy-L-larginine (MLA) is known to be a general nitric oxide synthase (NOS) inhibitor capable of inhibiting the inducible (iNOS), endothelial (eNOS) and neuronal isoforms (nNOS) of NOS. MLA is capable of being taken up into cells where it inhibits nitric oxide synthases. For this experiment, a 7-mer polymer of N^{G}-monomethy-L-larginine (MLA) was generated in order to assess the effect of creating a very large polymer of the general nitric oxide inhibitor in order to assess if it were capable of being absorbed or otherwise taken up by endothelial cells in-vivo due to its size.

First, peritoneal exudate cells were elicited by thioglycolate. These cells were then harvested and cultured in 96 well microtiter wells at 1×10⁶ overnight in DMEM media with 5% FCS. Media was then replaced with DMEM containing 50 Units/ml of IFNγ and 100 ng/ml bacterial lipopolysaccharide. Serial dilutions of either N^{G}-monomethy-L-larginine (MLA) or Polymethylarginine (poly-MLA 7-mer) were then added to experimental wells. The highest concentration of MLA was 1 mM and of Polymethylarginine was 400 uM.

The cultures were then incubated for 48 hours and culture supernatants were assayed for the NO metabolite nitrate using the Griess reaction after nitrate reduction. The positive control and the lowest dilutions of MLA and poly-MLA demonstrated over 100 μM nitrite synthesis. Serial dilutions of both inhibitors blocked NO production, with poly-MLA exhibiting slightly greater inhibition. These results indicate that poly-MLA is taken up into endothelial cells by direct contact and that it appears to be functional in inhibiting NO synthesis within the endothelial cell after such uptake.

The compositions and methods disclosed and claimed herein can be made and executed without undue experimentation in light of the present disclosure. While the compositions and methods of this invention have been described in terms of particular embodiments, it will be apparent to those of skill in the art that variations may be applied to the compositions and methods and/or in the steps or in the sequence of steps of the methods described herein without departing from the concept, spirit and scope of the invention. More specifically, it will be apparent that certain related components may be substituted for the components described herein while the same or similar results would be achieved. All such similar substitutes and modifications apparent to those skilled in the art are deemed to be within the spirit, scope and concept of the invention as defined by the appended claims.

What is claimed is:

1. A composition for treating a disease or condition comprising:
a pharmaceutically effective amount of a compound that is directed to the endothelial cells of a mammal in order to inhibit nitric oxide synthase activity in said endothelial cells, wherein said compound that inhibits nitric oxide synthase activity is a modified general nitric oxide synthase inhibitor or an eNOS inhibitor.

2. The composition of claim 1, wherein said modified general nitric oxide synthase inhibitor is based on a molecule selected from arginine-based analogues, methylated arginines, substituted L-arginine, nitro-arginine, L-N^{G}-nitroarginine, N^{G}-mono-methyl-L-arginine (L-NMMA), N-nitro-L-arginine methyl ester (L-NAME), N-amino-L-arginine, N-methyl-L-arginine, N^{G}-monomethyl-L-arginine (L-NMA), N^{G}-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, or mixtures thereof.

3. The composition of claim 1, wherein said modified general nitric oxide inhibitor includes the conversion into a polymeric form or conjugation to polyethylene glycol.

4. The composition of claim 3, wherein said polymeric form is (N^{G}-mono-methyl-L-arginine)⁷.

5. The composition of claim 3, wherein said general nitric oxide inhibitor conjugated to polyethylene glycol is N^{G}-mono-methyl-L-arginine.

6. The composition of claim 1, wherein said eNOS inhibitor is selected from asymmetric dimethylarginine or N(5)-(1-iminoethyl)-L-ornithine (L-NIO).

7. The composition of claim 1, further comprising the administration of a pharmaceutically effective amount of a nitric oxide scavenger.

8. The method according to claim 1, wherein said disease or condition is selected from hypotension, septic shock, vascular leak syndrome, cancer, sepsis, acidosis, spinal anesthesia, administration of inhalation agents, administration of nitrate preparations, administration of calcium channel blockers or administration of ACE inhibitors.

9. The composition of claim 1, further comprising a pharmaceutically effective amount of an interleukin to said mammal.

10. The composition of claim 9, wherein said interleukin is interleukin-2.

11. A method for treating a disease or condition comprising:
administering a pharmaceutically effective amount of a compound that is directed to the endothelial cells of a mammal in order to inhibit nitric oxide synthase activity in said endothelial cells, wherein said compound that inhibits nitric oxide synthase activity is a modified general nitric oxide synthase inhibitor or an eNOS inhibitor.

12. The method of claim 11, wherein said modified general nitric oxide synthase inhibitor is based on a molecule selected from arginine-based analogues, methylated arginines, substituted L-arginine, nitro-arginine, L-N^{G}-nitroarginine, N^{G}-mono-methyl-L-arginine (L-NMMA), N-nitro-L-arginine methyl ester (L-NAME), N-amino-L-arginine, N-methyl-L-arginine, N^{G}-monomethyl-L-arginine (L-NMA), N^{G}-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, or mixtures thereof.

13. The method of claim 11, wherein said modified general nitric oxide inhibitor includes the conversion into a polymeric form or conjugation to polyethylene glycol.

14. The method of claim 13, wherein said polymeric form is (N^{G}-mono-methyl-L-arginine)⁷.

15. The method of claim 13, wherein said general nitric oxide inhibitor conjugated to polyethylene glycol is N^{G}-mono-methyl-L-arginine.

16. The method of claim 11, wherein said eNOS inhibitor is asymmetric dimethylarginine or N(5)-(1-iminoethyl)-L-ornithine (L-NIO).

17. The method of claim 11, wherein said disease or condition is selected from hypotension, septic shock, vascular leak syndrome, cancer, sepsis, acidosis, spinal anesthesia, administration of inhalation agents, administration of nitrate preparations, administration of calcium channel blockers or administration of ACE inhibitors.

18. The method of claim 11, further comprising the administration of a pharmaceutically effective amount of an agent selected from an interleukin or a nitric oxide scavenger.

19. The method of claim 18, wherein said pharmaceutically effective dose of interleukin is increased due to said inhibition of nitric oxide synthase activity in said endothelial cells.

20. The method of claim 18, wherein the toxic effects of said interleukin are decreased due to said inhibition of nitric oxide synthase activity in said endothelial cells.
